(19) United States
(12) Patent Application Publication
(10) Pub. No.: US 2024/0339176 A1
(43) Pub. Date: Oct. 10, 2024

(54) Artificial Intelligence Guided Production of Cells and Organs from Pluripotent Stem Cells

(71) Applicant: Creative Medical Technologies, Inc., Phoenix, AZ (US)

(72) Inventors: Thomas Ichim, San Diego, CA (US); Timothy Warbington, Phoenix, AZ (US); Amit Patel, Salt Lake City, UT (US); Courtney Bartlett, Niceville, FL (US)

(73) Assignee: Creative Medical Technologies, Inc., Phoenix, AZ (US)

(21) Appl. No.: 18/630,510

(22) Filed: Apr. 9, 2024

Related U.S. Application Data
(60) Provisional application No. 63/458,423, filed on Apr. 10, 2023.

(51) Int. Cl. G16B 40/00; G06N 5/022

(52) U.S. Cl. CPC G16B 40/00; G06N 5/022

(57) ABSTRACT

Disclosed are systems, means and compositions of matter utilizing artificial intelligence to create unique cells and/or organs from pluripotent stem cells. In one embodiment an artificial intelligence/machine learning approach is utilized to overview and categorize molecular and cellular data regarding normal embryonic development and associated pathways. Through acquiring this information, said artificial intelligence/machine learning system develops a graded list of morphogens/differentiating agents and/or conditions that are utilized to replicate the process of cell/tissue/organ formation artificially. In one embodiment the invention teaches generation of artificial pancreatic organoids through growth factors predicted by said artificial intelligence/machine learning systems. In other embodiments embryogenesis is recapitulated in adult tissue using predicted morphogens and/or extracellular matrix treatments.

### CROSS-REFERENCE TO RELATED APPLICATIONS

The present application claims benefit of Provisional Patent Application Ser. No. 63/458,423, filed on Apr. 10, 2023, titled ARTIFICIAL INTELLIGENCE GUIDED PRODUCTION OF CELLS AND ORGANS FROM PLURIPOTENT STEM CELLS, the contents of which are incorporated herein by reference in its entirety.

### FIELD OF THE INVENTION

The teachings herein related to the use of artificial intelligence to generate cells and organs from pluripotent stem cells.

### BACKGROUND OF THE INVENTION

Numerous techniques are currently employed for differentiation of stem cells into various tissues, these primarily include methods based on replicating embryological development. Unfortunately, current means of manipulation stem cells differentiation are limited to the number of cells generated, as well as the quality of cells.

The utilization of deep learning technologies allows for rapid in silico screening of compounds to assess multiple biological activities, including new uses of existing drugs. In the current patent the utilization of stem cell associated pathways is leveraged to all for use of deep learning algorithms to instruct the repositioning of drugs which are already approved, as well as pipeline candidates.

### SUMMARY OF THE INVENTION

Preferred methods are directed to embodiments wherein computer-implemented method for identifying agents capable of inducing specific differentiation of pluripotent stem cells into a particular tissue through the utilization of a computing system, said comprising: a) obtaining information regarding cellular and molecular characteristics of an embryonic developing tissue; b) analyzing one or more molecular and/or cellular features of the tissue during stages of differentiation, wherein analyzing the one or more features includes using an artificial intelligence architecture that includes a separable convolutional neural network; and based at least in part on the analysis of the one or more features; and c) obtaining predictive data in the form of associated proteins, extracellular matrix, and nucleic acids that are associated with the process of differentiation.

Preferred methods include embodiments enabled/designed to create a score for each of the one or more methodologies and/or molecular pathways associated with cellular differentiation, wherein the score reflects the likelihood that an said pathway and/or methodology contains a the necessary information and/or detail to reproducibly generate differentiation of said pluripotent stem cells into a desired tissue and/or organ.

Preferred methods include embodiments further comprising indicating whether the one or more molecular pathways and/or methodologies have synergy and/or antagonism.

Preferred methods include embodiments further comprising segregating said methodologies and/or molecular pathways into various bioinformatics based categories.

Preferred methods include embodiments wherein said bioinformatics based categories include ras associated pathways.

Preferred methods include embodiments wherein said bioinformatics based categories include notch associated pathways.

Preferred methods include embodiments wherein said bioinformatics based categories include c-myc associated pathways.

Preferred methods include embodiments wherein said bioinformatics based categories include BMP associated pathways.

Preferred methods include embodiments wherein said bioinformatics based categories include hedgehog associated pathways.

Preferred methods include embodiments wherein said bioinformatics based categories include NF-kappa associated pathways.

Preferred methods include embodiments wherein said bioinformatics based categories include Fas associated pathways.

Preferred methods include embodiments wherein said bioinformatics based categories include TRAF associated pathways.

Preferred methods include embodiments wherein said bioinformatics based categories include TRAIL associated pathways.

Preferred methods include embodiments wherein said bioinformatics based categories include TNF-alpha associated pathways.

Preferred methods include embodiments wherein said bioinformatics based categories include c-met associated pathways.

Preferred methods include embodiments wherein said bioinformatics based categories include endoglin associated pathways.

Preferred methods include embodiments wherein said bioinformatics based categories include PDX-1 associated pathways.

Preferred methods include embodiments wherein said bioinformatics based categories include IL-3 associated pathways.

Preferred methods include embodiments wherein the one or more molecular pathways associated with differentiation to a specific tissue comprises a dataset of differentiating factors which are corresponding to a biological sample taken from a single subject.

Preferred methods include embodiments wherein indicating whether the one or more molecular pathways associated with differentiation further comprises identification of the concentration of differentiation factors/or the combination of differentiating factors with de-differentiation promoting factors.

Preferred methods include embodiments wherein said pluripotent stem cell is an inducible pluripotent stem cell.

Preferred methods include embodiments wherein said pluripotent stem cell is an inducible pluripotent stem cell.

Preferred methods include embodiments wherein said inducible pluripotent stem cell is generated by a mechanical stress associated means.

Preferred methods include embodiments wherein said generation of said inducible pluripotent stem cell by a mechanical stress associated means involves a process comprising the step of: applying a mechanical stress to a somatic cell to produce mechanical strain that induces the somatic cell to become a pluripotent stem cell.

Preferred methods include embodiments wherein said somatic cell is predisposed to dedifferentiation.

Preferred methods include embodiments wherein said somatic cell is predisposed to dedifferentiation is a thymus derived cell.

Preferred methods include embodiments wherein said thymus derived cell expresses the marker CD3.

Preferred methods include embodiments wherein said thymus derived cell expresses the marker CD28.

Preferred methods include embodiments wherein said thymus derived cell expresses the marker CTLA4.

Preferred methods include embodiments wherein said thymus derived cell expresses the marker ICOS.

Preferred methods include embodiments wherein said thymus derived cell expresses the marker CD25.

Preferred methods include embodiments wherein said thymus derived cell expresses the marker recombinase associated gene 1.

Preferred methods include embodiments wherein said thymus derived cell expresses the marker recombinase associated gene 2.

Preferred methods include embodiments wherein said thymus derived cell expresses foxp3.

Preferred methods include embodiments wherein said thymus derived cell has been

treated with a histone deacetylase inhibitor.

Preferred methods include embodiments wherein said histone deactylase inhibitor is vorinostat.

Preferred methods include embodiments wherein said histone deactylase inhibitor is entinostat.

Preferred methods include embodiments wherein said histone deactylase inhibitor is panobinostat.

Preferred methods include embodiments wherein said histone deactylase inhibitor is trichostatin A.

Preferred methods include embodiments wherein said histone deactylase inhibitor is mocetinostat.

Preferred methods include embodiments wherein said histone deactylase inhibitor is belinostat.

Preferred methods include embodiments wherein said histone deactylase inhibitor is romidepsin.

Preferred methods include embodiments wherein said histone deactylase inhibitor is MC1568.

Preferred methods include embodiments wherein said histone deactylase inhibitor is tubastatin.

Preferred methods include embodiments wherein said histone deactylase inhibitor is givinostat.

Preferred methods include embodiments wherein said histone deactylase inhibitor is dacinostat.

Preferred methods include embodiments wherein said histone deactylase inhibitor is CUDC1.

Preferred methods include embodiments wherein said histone deactylase inhibitor is quisinostat.

Preferred methods include embodiments wherein said histone deactylase inhibitor is parcinostat.

Preferred methods include embodiments wherein said histone deactylase inhibitor is PCI-34151.

Preferred methods include embodiments wherein said histone deactylase inhibitor is troxinostat.

Preferred methods include embodiments wherein said histone deactylase inhibitor is anexinostat.

Preferred methods include embodiments wherein said histone deactylase inhibitor is RFGP96.

Preferred methods include embodiments wherein said histone deactylase inhibitor is AR42.

Preferred methods include embodiments wherein said histone deactylase inhibitor is ricolinostat.

Preferred methods include embodiments wherein said histone deactylase inhibitor is CI994.

Preferred methods include embodiments wherein said histone deactylase inhibitor is Finpinostat.

Preferred methods include embodiments wherein said histone deactylase inhibitor is M344.

Preferred methods include embodiments wherein said histone deactylase inhibitor is tubacin.

Preferred methods include embodiments wherein said histone deactylase inhibitor is RG2833.

Preferred methods include embodiments wherein said histone deactylase inhibitor is resminostat.

Preferred methods include embodiments wherein said histone deactylase inhibitor is tubastatin A.

Preferred methods include embodiments wherein said histone deactylase inhibitor is KT531.

Preferred methods include embodiments wherein said histone deactylase inhibitor is KA2507.

Preferred methods include embodiments wherein said histone deactylase inhibitor is ACY775.

Preferred methods include embodiments wherein said histone deactylase inhibitor is Tubastatin TFA.

Preferred methods include embodiments wherein said histone deactylase inhibitor is BRD3308.

Preferred methods include embodiments wherein said histone deactylase inhibitor is SIS17.

Preferred methods include embodiments wherein said histone deactylase inhibitor is SR3470.

Preferred methods include embodiments wherein said histone deactylase inhibitor is TC H106.

Preferred methods include embodiments wherein said histone deactylase inhibitor is NKL22.

Preferred methods include embodiments wherein said histone deactylase inhibitor is EDO-S101.

Preferred methods include embodiments wherein said histone deactylase inhibitor is SKLB-23bb.

Preferred methods include embodiments wherein said histone deactylase inhibitor is TH34.

Preferred methods include embodiments wherein said histone deactylase inhibitor is suberohydroxamic acid.

Preferred methods include embodiments wherein said histone deactylase inhibitor is UF010.

Preferred methods include embodiments wherein said histone deactylase inhibitor is WT161.

Preferred methods include embodiments wherein said histone deactylase inhibitor is UF010.

Preferred methods include embodiments wherein said histone deactylase inhibitor is valproic acid.

Preferred methods include embodiments wherein said histone deactylase inhibitor is ACY738.

Preferred methods include embodiments wherein said histone deactylase inhibitor is chidamide.

Preferred methods include embodiments wherein said histone deactylase inhibitor is TMP195.

Preferred methods include embodiments wherein said histone deactylase inhibitor is citarinostat.

Preferred methods include embodiments wherein said histone deactylase inhibitor is BRD73954.

Preferred methods include embodiments wherein said histone deactylase inhibitor is bg45.

Preferred methods include embodiments wherein said histone deactylase inhibitor is domatinostat.

Preferred methods include embodiments wherein said histone deactylase inhibitor is CAY10603.

Preferred methods include embodiments wherein said histone deactylase inhibitor is LMK235.

Preferred methods include embodiments wherein said histone deactylase inhibitor is splitomicin.

Preferred methods include embodiments wherein said histone deactylase inhibitor is CAY10683.

Preferred methods include embodiments wherein said histone deactylase inhibitor is nexturastat A.

Preferred methods include embodiments wherein said histone deactylase inhibitor is TMP269.

Preferred methods include embodiments wherein said histone deactylase inhibitor is HBOP.

Preferred methods include embodiments wherein said histone deactylase inhibitor is sodium salt of valproic acid.

Preferred methods include embodiments wherein said histone deactylase inhibitor is sodium butyrate.

Preferred methods include embodiments wherein said histone deactylase inhibitor is curcumin.

Preferred methods include embodiments wherein said histone deactylase inhibitor is divalprox sodium.

Preferred methods include embodiments wherein said histone deactylase inhibitor is striptaid.

Preferred methods include embodiments wherein said histone deactylase inhibitor is sodium phenylbutyrate.

Preferred methods include embodiments wherein said histone deactylase inhibitor is 4-PBA.

Preferred methods include embodiments wherein said histone deactylase inhibitor is GSK3117391.

Preferred methods include embodiments wherein said histone deactylase inhibitor is sulforaphane.

Preferred methods include embodiments wherein said histone deactylase inhibitor is raddeanin.

Preferred methods include embodiments wherein said histone deactylase inhibitor is isoguanasine.

Preferred methods include embodiments wherein said histone deactylase inhibitor is sinapinic acid.

Preferred methods include embodiments wherein said histone deactylase inhibitor is tasquinimod.

Preferred methods include embodiments wherein said histone deactylase inhibitor is parthenolide.

Preferred methods include embodiments one of the pluripotency associated genes is transfected into said cell to which mechanical stress is to be added.

Preferred methods include embodiments wherein said pluripotency associated gene is the octamer-binding transcription factor 4 (OCT4).

Preferred methods include embodiments wherein said pluripotency associated gene is the homeobox associated protein NANOG.

Preferred methods include embodiments wherein said pluripotency associated gene is

the sex determining region Y-box 2 (SOX-2).

Preferred methods include embodiments wherein said cell to be converted to a pluripotent stem cell by said mechanical stress associated means is plated in a droplet that partially or fully surrounds said somatic cell.

Preferred methods include embodiments wherein forming the droplet includes thermal inkjet printing.

Preferred methods include embodiments wherein forming the droplet includes pressurized flow through an orifice without heat.

Preferred methods include embodiments wherein forming the droplet includes pressurized flow through an orifice without heat but with increased atmospheric pressure compared to baseline conditions.

Preferred methods include embodiments said increased atmospheric pressure is sufficient to induce activation of mitogen activated protein kinase.

Preferred methods include embodiments said increased atmospheric pressure is sufficient to induce activation of inhibitor of kappa b.

Preferred methods include embodiments said increased atmospheric pressure is sufficient to induce activation of janus activated kinase.

Preferred methods include embodiments said increased atmospheric pressure is sufficient to induce activation of NF-kappa B.

Preferred methods include embodiments said increased atmospheric pressure is sufficient to induce upregulation of leukemia inhibitory factor receptor.

Preferred methods include embodiments said increased atmospheric pressure is sufficient to induce activation of SSEA4.

Preferred methods include embodiments said increased atmospheric pressure is sufficient to induce activation of SSEA3.

Preferred methods include embodiments said increased atmospheric pressure is sufficient to induce activation of NANOG.

Preferred methods include embodiments said increased atmospheric pressure is sufficient to induce activation of c-mpl.

Preferred methods include embodiments said increased atmospheric pressure is sufficient to induce activation of TRA-1.

Preferred methods include embodiments said increased atmospheric pressure is sufficient to induce activation of c-kit.

Preferred methods include embodiments said increased atmospheric pressure is sufficient to induce activation of GDF-9.

Preferred methods include embodiments said increased atmospheric pressure is sufficient to induce activation of GDF-11.

Preferred methods include embodiments said increased atmospheric pressure is sufficient to induce activation of HDAC6.

Preferred methods include embodiments said increased atmospheric pressure is sufficient to induce activation of NOBOX.

Preferred methods include embodiments said increased atmospheric pressure is sufficient to induce activation of DAZL.

Preferred methods include embodiments said increased atmospheric pressure is sufficient to induce activation of Stella.

Preferred methods include embodiments said increased atmospheric pressure is sufficient to increase production of interleukin 1 beta.

Preferred methods include embodiments said increased atmospheric pressure is sufficient to increase production of interleukin 1 receptor antagonist.

Preferred methods include embodiments said increased atmospheric pressure is sufficient to increase production of interleukin-3.

Preferred methods include embodiments said increased atmospheric pressure is sufficient to increase production of interleukin-6.

Preferred methods include embodiments said increased atmospheric pressure is sufficient to increase production of interleukin-8.

Preferred methods include embodiments said increased atmospheric pressure is sufficient to increase production of interleukin-10.

Preferred methods include embodiments said increased atmospheric pressure is sufficient to increase production of G-CSF.

Preferred methods include embodiments said increased atmospheric pressure is sufficient to increase production of M-CSF.

Preferred methods include embodiments said increased atmospheric pressure is sufficient to increase production of GM-CSF.

Preferred methods include embodiments said increased atmospheric pressure is sufficient to increase production of GC-MAF.

Preferred methods include embodiments said increased atmospheric pressure is sufficient to increase production of lymphotoxin.

Preferred methods include embodiments said increased atmospheric pressure is sufficient to increase production of MCP.

Preferred methods include embodiments said increased atmospheric pressure is sufficient to increase production of MIP-18 alpha.

Preferred methods include embodiments said increased atmospheric pressure is sufficient to increase production of MIP-1 beta.

Preferred methods include embodiments said increased atmospheric pressure is sufficient to increase production of placental growth factor.

Preferred methods include embodiments said increased atmospheric pressure is sufficient to increase production of angiopoietin.

Preferred methods include embodiments said increased atmospheric pressure is sufficient to increase production of endoglin.

Preferred methods include embodiments said increased atmospheric pressure is sufficient to increase production of notch.

Preferred methods include embodiments said increased atmospheric pressure is sufficient to increase production of jagged.

Preferred methods include embodiments said increased atmospheric pressure is sufficient to increase production of wnt1.

Preferred methods include embodiments said increased atmospheric pressure is sufficient to increase production of GDF-11.

Preferred methods include embodiments wherein said cell to be converted to a pluripotent stem cell by said mechanical stress associated means is plated in a droplet, wherein forming the droplet includes laser induced fast forward transfer.

Preferred methods include embodiments wherein forming the droplet includes using a piezoelectric transducer.

Preferred methods include embodiments wherein forming the droplet includes laser bioprinting.

Preferred methods include embodiments wherein forming the droplet includes using an ultrasonic transducer.

Preferred methods include embodiments wherein forming the droplet includes using at least one microvalve.

Preferred methods include embodiments further comprising printing the pluripotent stem cell on a substrate and incubating the pluripotent stem cell.

Preferred methods include embodiments wherein said substrate is a decellularized matrix.

Preferred methods include embodiments wherein said decellularized matrix is obtained from amniotic membrane.

Preferred methods include embodiments wherein said decellularized matrix is obtained from bone marrow.

Preferred methods include embodiments wherein said decellularized matrix is obtained from omental tissue.

Preferred methods include embodiments wherein said decellularized matrix is obtained from intestinal mucosal tissue.

Preferred methods include embodiments wherein said decellularized matrix is obtained from fallopian tube tissue.

Preferred methods include embodiments wherein said decellularized matrix is obtained from endometrial tissue.

Preferred methods include embodiments wherein said decellularized matrix is obtained from placental tissue.

Preferred methods include embodiments wherein said decellularized placental membrane is prepared by obtaining an amniotic sac cut from a placenta around a placenta body, wherein the amniotic sac comprises a pre-decellularized placental membrane and the pre-decellularized placental membrane comprises an amnion layer and a chorion layer.

Preferred methods include embodiments wherein the pre-decellularized placental membrane is treated with N-lauroylsarcosinate.

Preferred methods include embodiments wherein the pre-decellularized placental membrane is treated with n-octyl-b-D-glucopyranoside.

Preferred methods include embodiments wherein the pre-decellularized placental membrane is treated with polyoxyethylene alcohol.

Preferred methods include embodiments wherein the pre-decellularized placental membrane is treated with polyoxyethylene isoalcohol.

Preferred methods include embodiments wherein the pre-decellularized placental membrane is treated with polyoxyethylene p-t-octyl phenol.

Preferred methods include embodiments wherein the pre-decellularized placental membrane is treated with polyoxyethylene nonyphenol.

Preferred methods include embodiments wherein the pre-decellularized placental membrane is treated with a or more polyoxyethylene ester of a fatty acid.

Preferred methods include embodiments wherein cells are removed from pre-decellularized placental membrane without separating the amnion layer from the chorion layer

Preferred methods include embodiments wherein the decellularized placental membrane comprises one or more growth factors in an amount that is at least 10% greater than the sum of the amount of the one or more growth factors in a decellularized control isolated amniotic membrane having the same size as the decellularized placental membrane and the amount of the one or more growth factors in a decellularized control isolated chorionic membrane having the same size as the decellularized placental membrane, wherein the decellularized control isolated amniotic membrane is prepared by removing cells from the amnion layer isolated from the pre-decellularized placental membrane in the same manner as the decellularized placental membrane is prepared from the pre-decellularized placental membrane, and wherein the decellularized control isolated chorion is prepared by removing cells from the chorion layer isolated from the pre-decellularized placental membrane in the same manner as the decellularized placental membrane is prepared from the pre-decellularized placental membrane.

Preferred methods include embodiments wherein the decellularized placental membrane comprises one or more growth factors in an amount that is at least 20% of the amount of the one or more growth factors in the pre-decellularized placental membrane.

Preferred methods include embodiments wherein said growth factor is HGF-1.

Preferred methods include embodiments wherein said growth factor is acidic fibroblast growth factor.

Preferred methods include embodiments wherein said growth factor is basic fibroblast growth factor.

Preferred methods include embodiments wherein said growth factor is keratinocyte derived growth factor.

Preferred methods include embodiments wherein said growth factor is nerve growth factor.

Preferred methods include embodiments wherein said growth factor is brain derived neurotrophic growth factor.

Preferred methods include embodiments wherein said growth factor is ciliary trophic nerve growth factor.

Preferred methods include embodiments wherein said growth factor is transforming growth factor alpha.

Preferred methods include embodiments wherein said growth factor is transforming growth factor beta.

Preferred methods include embodiments wherein said growth factor is epidermal growth factor.

Preferred methods include embodiments wherein said growth factor is vascular endothelial growth factor.

Preferred methods include embodiments wherein said growth factor is interleukin-3.

Preferred methods include embodiments wherein said growth factor is interleukin-4.

Preferred methods include embodiments wherein said growth factor is interleukin-6.

Preferred methods include embodiments wherein said growth factor is interleukin-8.

Preferred methods include embodiments wherein said growth factor is interleukin-10.

Preferred methods include embodiments wherein said growth factor is interleukin-11.

Preferred methods include embodiments wherein said growth factor is interleukin-13.

Preferred methods include embodiments wherein said growth factor is interleukin-16.

Preferred methods include embodiments wherein said growth factor is interleukin-20.

Preferred methods include embodiments wherein said growth factor is interleukin-22.

Preferred methods include embodiments wherein said growth factor is interleukin-27.

Preferred methods include embodiments wherein said decellularized matrix is

decellularized placental membrane comprises less than 100 ng dsDNA per mg dry weight of the decellularized placental membrane.

Preferred methods include embodiments wherein the decellularized placental

membrane comprises DNA in an amount that is less than 10% of the DNA in the pre-decellularized placental membrane.

The computed implemented method of claim 168, wherein the amnion layer in the pre-decellularized placental membrane comprises a fibroblast layer, and wherein the chorion layer in the pre-decellularized placental membrane comprises a reticular layer.

The computed implemented method of claim 168, wherein the amnion layer in the pre-decellularized placental membrane comprises epithelium, a basement membrane, a compact layer, a fibroblast layer, and a spongy layer, and wherein the chorion layer in the pre-decellularized placental membrane comprises a cellular layer, a reticular layer, a pseudo-basement membrane, and a trophoblast layer.

Preferred methods include embodiments wherein said placental matrix is treated win a Nobel gas prior to utilization.

Preferred methods include embodiments wherein said Nobel gas is xenon.

Preferred methods include embodiments wherein said Nobel gas is argon.

Preferred methods include embodiments further comprising harvesting the amniotic sac from a donor.

Preferred methods include embodiments further comprising cleaning and disinfecting the pre-decellularized placental membrane.

Preferred methods include embodiments further comprising fixing the pre-decellularized placental membrane onto a frame before the cell removal step.

Preferred methods include embodiments wherein the chorion layer in the pre-decellularized placental membrane comprises a trophoblast layer.

Preferred methods include embodiments wherein the one or more compounds are present in the composition at a concentration of 0.01-5% (w/v).

Preferred methods include embodiments further comprising treating the pre-decellularized placental membrane with one or more endonucleases at a concentration of 20-400 U/mL.

### DETAILED DESCRIPTION OF THE INVENTION

The invention teaches the identification of biological pathways associated with stem cell healing of diseased or degenerated tissues and leveraging knowledge of these pathways to screen for drugs to activate such pathways. This provides means of repositioning drugs that are FDA cleared, as well as finding new indications for therapeutics in the drug development pipeline. Such therapeutics may refer to a branch of medicine concerned with the treatment of disease and the action of remedial agents (e.g., drugs). Therapeutics includes, but is not limited to, the field of general pharmaceuticals. Entities in the therapeutics industry may discover, develop, produce, and market drugs for use as medications to be administered or self-administered to patients. Goals of administering or self-administering the drugs may include curing the patient of a disease, causing an active disease to enter a state of remission, vaccinating the patient by stimulating the immune system to better protect against the disease, and/or alleviating, mitigating or ameliorating a symptom. Existing drug discoveries may be based on any combination of human design, high-throughput screening, synthetic products, natural substances and various types of cellular therapeutics.

As used herein, the term “AI-designed molecule” is used to refer to a molecule that was designed, generated, or otherwise developed using one or more machine learning (ML) and/or AI techniques. These molecules are used for differentiation of stem cells and/or expansion of stem cells. The disclosed AI-designed molecules can include biological molecules (e.g., natural and recombinant peptides, proteins, biopolymers, nucleic acids, polysaccharides, antibodies, hormones, etc.), synthetic molecules, biopharmaceuticals (or “biologics”), and combinations thereof. The disclosed AI-designed molecules can include organic compounds, inorganic compounds, organometallic compounds, or combinations thereof.

The term “peptide” as used herein refers to a polymer of amino acid residues typically ranging in length from 2 to about 50 residues. In certain embodiments the AI-designed peptides disclosed herein range from about 2 to 25 residues in length. In some embodiments the amino acid residues comprising the peptide are “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers. In some embodiments of the invention, AI based algorithms are utilized to screen peptides for modulation of stem cell differentiation inducing activity. As used herein, the term “synthetic” peptide is used to refer to a peptide that is chemically synthesized as opposed to host derived. The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. In various embodiments, the disclosed AI-designed peptides comprise only of natural amino acid residues. In some embodiments, the disclosed AI-designed molecules can substitute one or more synthetic or modified amino acids for a corresponding natural amino acid. A compound of the invention “corresponds” to a natural peptide if it elicits a biological activity (e.g., stem cell differentiation activity) related to the biological activity and/or specificity of the naturally occurring peptide. The elicited activity may be the same as, greater than or less than that of the natural peptide. In general, such a peptide will have an essentially corresponding monomer sequence, where a natural amino acid is replaced by an N-substituted glycine derivative, if the N-substituted glycine derivative resembles the original amino acid in hydrophilicity, hydrophobicity, polarity, etc. It should further be appreciated that the disclosed peptides can include the primary sequences disclosed herein, and conservatively modified variants thereof.

In certain embodiments, some modifications of naturally occurring peptides are used, where said modifications are compromising at least 80%, preferably at least 85% or 90%, and more preferably at least 95% or 98% sequence identity with any of the sequences described herein are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides disclosed herein sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using a basic local alignment search tool (BLAST) or the like.

The term “specificity” when used with respect to the antimicrobial activity of a peptide indicates that the peptide preferentially inhibits growth and/or proliferation and/or exterminates a particular microbial species as compared to other related species. In certain embodiments the preferential inhibition or exterminate is at least 10% greater (e.g., the LD50 being 10% lower), preferably at least 20%, 30%, 40%, or 50%, more preferably at least 2-fold, at least 5-fold, or at least 10-fold greater for the target species.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “high” as used with respect to stem cell differentiation activity and/or potency is used herein to indicate that the level of antimicrobial activity of an antimicrobial agent (e.g., an AMP or the like) is greater than a defined minimum threshold of stem cell differentiation activity or potency for a particular cellular type.

The term “low-toxicity” is used herein to indicate any level of toxicity of a pharmacological agent that is less than defined acceptable threshold of toxicity. In various embodiments, the defined threshold can be based on the MIC of the pharmacological agent relative to its LD50 and/or HC50 concentration. In some implementations, a pharmacological agent (e.g., a stem cell differentiation stimulator) can be considered to have low-toxicity if its MIC is less than its LD50 and/or HC50 concentration. In other implementations, a pharmacological agent can be considered to have low-toxicity if its MIC is 60% or less than its LD50 and/or HC50 concentration. In other implementations, a pharmacological agent can be considered to have low-toxicity if its MIC is 50% or less than its LD50 and/or HC50 concentration. In other implementations, a pharmacological agent can be considered to have low-toxicity if its MIC is 30% or less than its LD50 and/or HC50 concentration. In other implementations, a pharmacological agent can be considered to have low-toxicity if its MIC is 25% or less than its LD50 and/or HC50 concentration.

In one embodiment, the invention teaches the utilization of databases containing mechanism of action pathways, identifying drugs for repositioning from said pathways, and assessing in silico, or in vitro or in vivo the chosen candidates for modulation. In one embodiment candidates are screened for ability to alter CD markers, said CD markers are proteins termed “cluster of differentiation” proteins. In the area of stem cells, one embodiment of the invention is the use of machine learning algorithms, either supervised or unsupervised, for selection of agents to manipulate molecules such as CXCR4 which can be useful to enhance homing of stem cells, particularly, but not limited to, stem cells of the mesenchymal lineage.

The invention teaches means of overcoming existing hurdles in the area of drug development. For example, it is generally accepted in the field of pharmaceutical sciences that conventional drug discoveries are based on human design, high-throughput screening, and/or natural substances may be inefficient, riven with noise, limited in application, not efficacious, dangerous or poisonous, and/or not defensible. Further, in some instances, there are instances of certain diseases (e.g., instances of prosthetic joint infections) that do not have a corresponding existing therapeutic to treat the certain diseases or which provide temporary results against which the disease is refractory. One reason for the lack of an existing therapeutic may be the conventional drug discovery techniques are incapable of discovering the therapeutic needed to treat the certain diseases. By “treat,” we mean that the disease at hand is cured inter alia, that it is not refractory to treatment. The amount of knowledge, data, assumptions, and queries used to discover a therapeutic to treat the certain disease may be unattainable, overwhelming, and/or inefficiently determined, such that conventional drug discovery techniques cannot overcome these obstacles. Improvement is desired in the field of therapeutics.

Through the integration of stem cells and artificial intelligence technologies, such as supervised and unsupervised deep learning, the invention provides means of rapidly scanning databases, as well as utilizing PCA and other forms of analysis to predict which existing compounds may modulate desired activities in stem cells, as well as how to design around existing compounds that provide a signal of efficacy but may have some other properties that are not desirable.

In some embodiments of the invention, induced pluripotent stem cells are utilized for screening of compounds predicted by said algorithms. Accordingly, aspects of the present disclosure generally relate to an artificial intelligence engine for generating candidate drugs for stem cell manipulation. By using various encoding types that enable performing searches in the design space in an efficient manner, the artificial intelligence engine (AI) may enlarge the design space to include the combination of drug information (e.g., structural, physical, semantic, activity, sequence, chemical, etc.). The architecture of the AI engine may include various computational techniques that reduce the computational complexity of using a large design space, thereby saving computing resources (e.g., reducing computing time, reducing processing resources, reducing memory resources, etc.). At the same time, the disclosed architecture may generate superior candidate drugs that include desirable features (e.g., structure, semantics, activity, sequence, clinical outcomes, etc.) found in the larger design space as compared to conventional techniques using the smaller design space. The artificial intelligence (AI) engine may use a combination of rational algorithmic discovery and machine learning models (e.g., generative deep learning methods) to produce enhanced therapeutics that may treat any suitable target disease and/or medical condition. The AI engine may discover, translate, design, generate, create, develop, formulate, classify, and/or test candidate drug compounds that exhibit desired activity (e.g., antimicrobial, immunomodulatory, cytotoxic, neuromodulatory, etc.) in design spaces for target diseases and/or medical conditions. Such candidate drug compounds that exhibit desired activity in a design space may effectively treat the disease and/or medical condition associated with that design space. In some embodiments, a selected candidate drug compound that effectively treats the disease and/or medical condition may be formulated into an actual drug for administration and may be tested in a lab and/or at a clinical stage. In general, the disclosed embodiments may enable rationally discovery of drug compounds for a larger design space at a larger scale, higher accuracy, and/or higher efficiency than conventional techniques. The AI engine may use various machine learning models to discover, translate, design, generate, create, develop, formulate, classify, and/or test candidate drug compounds. Each of the various machine learning models may perform certain specific operations. The types of machine learning models may include various neural networks that perform deep learning, computational biology, and/or algorithmic discovery. Examples of such neural networks may include generative adversarial networks, recurrent neural networks, convolutional neural networks, fully connected neural networks, etc., as described further below; and such networks may also additionally employ methods of or incorporating causal inference, including counterfactuals, in the process of discovery.

In some embodiments, a biological context representation of a set of drug compounds may be generated. The biological context representation may be a continuous representation of a biological setting that is updated as knowledge is acquired and/or data is updated. The biological context representation may be stored in a first data structure having a format (e.g., a knowledge graph) that includes both various nodes pertaining to health artifacts and various relationships connecting the nodes. The nodes and relationships may form logical structures having subjects and predicates. For example, one logical structure between two nodes having a relation may be “Genes are associated with Diseases” where “Genes” and “Diseases” are the subjects of the logical structure and “are associated with” is the relation. In such a way, the knowledge graph may encompass actual knowledge, rather than simply statistical inferences, pertaining to a biological setting. The information in the knowledge graph may be continuously or periodically updated and the information may be received from various sources curated by the AI engine. The knowledge in the biological context representation goes well beyond “dumb” data that just includes quantities of a value because the knowledge represents the relationships between or among numerous different types of data, as well as any or all of direct, indirect, causal, counterfactual or inferred relationships. In some embodiments, the biological context representation may not be stored, and instead, based on the stream of knowledge included in the biological context representation, may be streamed from data sources into the AI engine that generates the machine learning models. The biological context representation may be used to generate candidate drug compounds by translating the first data format to a second data structure having a second format (e.g., a vector). The second format may be more computationally efficient and/or suitable for generating candidate drug compounds that include sequences of ingredients that provide desired activity in a design space. “Ingredients” as used herein may refer, without limitation, to substances, compounds, elements, activities (such as the application or removal of electrical charge or a magnetic field for a specific maximum, minimum or discrete amount of time), and mixtures. Further, the second format may enable generating views of the levels of activity provided by the sequence of ingredients in a certain design space, as described further below.

At a high level, the AI engine may include at least one machine learning model that is trained to use causal inference to generate candidate drug compounds. One of the challenges with discovering new therapeutics may include determining whether certain ingredients are causal agents with respect to certain activity in a design space. The sheer number of possible sequences of ingredients may be extraordinarily large due to mathematical combinatorics, such that identifying a cause and effect relationship between ingredients and activity may be impossible or, at best, extremely unlikely, to identify without the disclosed embodiments. Based on advances in computing hardware (e.g., graphic processing unit processing cores) and the AI techniques using causal inference described herein, the disclosed embodiments may enable the efficient solving of the task of generating candidate drug compounds at scale.

By simulating numerous alternative scenarios to further optimize and hone the accuracy of a sequence of ingredients in the candidate drug compounds, such techniques may enable reducing the number of viable candidate drug compounds. As a result, the embodiments may provide technical benefits, such as reducing resources consumed (e.g., processing, memory, network bandwidth) by reducing a number of candidate drug compounds that may be considered for classification as a selected candidate drug compound by another machine learning model. In some embodiments, one application for the AI engine to design, discover, develop, formulate, create, and/or test candidate drug compounds may pertain to peptide therapeutics. A peptide may refer to a compound consisting of two or more amino acids linked in a chain. Example peptides may include dipeptides, tripeptides, tetrapeptides, etc. Aa polypeptide may refer to a long, continuous, and unbranched peptide chain. Peptides may be simple to manufacture at discovery scale, include drug-like characteristics of small molecules, include safety and high specificity of biologics, and/or provide greater administration flexibility than some other biologics.

Compounds may be tested in various stem cell differentiation assays as well as stem cell proliferation assays. The utilization of stem cells may involve hematopoietic stem cells, which typically express markers such as the adhesion molecule CD34, or other ones such as CD133 and/or CD105. In some cases modulation of marker expression is desired by the drugs being screened. For example, in some embodiments it is desired to increase homing of stem cells to a desired location, so increased CXCR4 is desired.

The disclosed techniques provide numerous benefits over conventional techniques for designing, developing, and/or testing candidate drug compounds for assessment in modulation of biological systems, in some embodiments for modulation of stem cell activity. For example, the AI engine may efficiently use a biological context representation of a set of drug compounds and one or more machine learning models to generate a set of candidate drug compounds and classify one of the set of candidate drug compounds as a selected candidate drug compound. Some embodiments may use causal inference to remove one or more potential candidate drug compounds from classification, thereby reducing the computational complexity and processing burden of classifying a selected candidate drug compound. In addition, benchmark analysis may be performed for each type of machine learning model that generates candidate drugs. The benchmark analysis may score various parameters of the machine learning models that generate the candidate drugs. The various parameters may refer to candidate drug novelty, candidate drug uniqueness, candidate drug similarity, candidate drug validity, etc. The scores may be used to recursively tune the machine learning models over time to cause one or more of the parameters to increase for the machine learning models. In some embodiments, some of the machine learning models may vary in their effectiveness as it pertains to some of the parameters. In addition, to generate subsequent candidate drug candidates, the benchmark analysis may score the candidate drug candidates generated by the machine learning models, rank the machine learning models that generate the highest scoring candidate drug candidates, and/or select the machine learning models producing the highest scoring candidate drug candidates.

Using causal inference, a generative adversarial network (GAN) may be used to generate a set of candidate drug compounds. A GAN refers to a class of deep learning algorithms including two neural networks, a generator and a discriminator, that both compete with one another to achieve a goal. For example, regarding candidate drug compound generation, the generator goal may include generating candidate drug compounds, including compatible/incompatible sequences of ingredients, and effective/ineffective sequences of ingredients, etc. that the discriminator classifies as feasible candidate drug compounds, including compatible and effective sequences of ingredients that may produce desired activity levels for a design space. In one embodiment, the generator may use causal inference, including counterfactuals, to calculate numerous alternative scenarios that indicate whether a certain result (e.g., activity level) still follows when any element or aspect of a sequence changes. For example, the generator may be a neural network based on Markov models (e.g., Deep Markov Models), which may perform causal inference. In some embodiments, one or more of the counterfactuals used during the causal inference may be determined and provided by the scientist module. The discriminator goal may include distinguishing candidate drug compounds which include undesirable sequences of ingredients from candidate drug compounds which include desirable sequences of ingredients. In some embodiments, the generator initially generates candidate drug compounds and continues to generate better candidate drug compounds after each iteration until the generator eventually begins to generate candidate drug compounds that are valid drug compounds which produce certain levels of activity within a design space. A candidate drug compound may be “valid” when it produces a certain level of effectiveness (e.g., above a threshold activity level as determined by a standard (e.g., regulatory entity)) in a design space. In order to classify the candidate drug compounds as a valid drug compound or invalid candidate drug compound, the discriminator may receive real drug compound information from a dataset and the candidate drug compounds generated by the generator. “Real drug compound,” as used in this disclosure, may refer to a drug compound that has been approved by any regulatory (governmental) body or agency. The generator obtains the results from the discriminator and applies the results in order to generate better (e.g., valid) candidate drug compounds. General details regarding the GAN are now discussed. The two neural networks, the generator and the discriminator, may be trained simultaneously. The discriminator may receive an input and then output a scalar indicating whether a candidate drug compound is an actual and/or viable drug compound. In some embodiments, the discriminator may resemble an energy function that outputs a low value (e.g., close to 0) when input is a valid drug compound and a positive value when the input is not a valid drug compound (e.g., if it includes an incorrect sequence of ingredients for certain activity levels pertaining to a design space). There are two functions that may be used, the generator function (G(V)), and the discriminator function (D(Y)). The generator function may be denoted as G (V), where V is generally a vector randomly sampled in a standard distribution (e.g., Gaussian). The vector may be any suitable dimension and may be referred to as an embedding herein. The role of the generator is to produce candidate drug candidates to train the discriminator function (D(Y)) to output the values indicating the candidate drug candidate is valid (e.g., a low value). During training, the discriminator is presented with a valid drug compound and adjusts its parameters (e.g., weights and biases) to output a value indicative of the validity of the candidate drug compounds that produce real activity levels in certain design spaces. Next, the discriminator may receive a modified candidate drug compound (e.g., modified using counterfactuals) generated by the generator and adjust its parameters to output a value indicative of whether the modified candidate drug compound provides the same or a different activity level in the design space. These can be compared to known agents which produce similar activities to regenerative cells or other biological systems.

The discriminator may use a gradient of an objective function to increase the value of the output. The discriminator may be trained as an unsupervised “density estimator,” i.e., a contrast function produces a low value for desired data (e.g., candidate drug compounds that include sequences producing desired levels of certain types of activity in a design space) and higher output for undesired data (e.g., candidate drug compounds that include sequences producing undesirable levels of certain types of activity in a design space). The generator may receive the gradient of the discriminator with respect to each modified candidate drug compound it produces. The generator uses the gradient to train itself to produce modified candidate drug compounds that the discriminator determines include sequences producing desired levels of certain types of activity in a design space. Recurrent neural networks include the functionality, in the context of a hidden layer, to process information sequences and store information about previous computations. As such, recurrent neural networks may have or exhibit a “memory.” Recurrent neural networks may include connections between nodes that form a directed graph along a temporal sequence. Keeping and analyzing information about previous states enables recurrent neural networks to process sequences of inputs to recognize patterns (e.g., such as sequences of ingredients and correlations with certain types of activity level). Recurrent neural networks may be similar to Markov chains. For example, Markov chains may refer to stochastic models describing sequences of possible events in which the probability of any given event depends only on the state information contained in the previous event. Thus, Markov chains also use an internal memory to store at least the state of the previous event. These models may be useful in determining causal inference, such as whether an event at a current node changes as a result of the state of a previous node changing.

The set of candidate drug compounds generated may be input into another machine learning model 132 trained to classify of the set of candidate drug compounds as a selected candidate drug compound. The classifier may be trained to rank the set of candidate drug compounds using any suitable ranking (i.e., for example, non-parametric) technique. For example, in some embodiments, one or more clustering techniques may be used to cluster the set of candidate drug compounds. To classify the selected candidate drug compound, the machine learning model 132 may also perform objective optimization techniques while clustering. To classify the selected candidate drug compound having desired levels of certain types of activity, the objective optimization may include using a minimization and/or maximization function for each candidate drug compound in the clusters. A cluster may refer to a group of data objects similar to one another within the same cluster, but dissimilar to the objects in the other clusters. Cluster analysis may be used to classify the data into relative groups (clusters). One example of clustering may include K-means clustering where “K” defines the number of clusters. Performing K-means clustering may comprise specifying the number of clusters, specifying the cluster seeds, assigning each point to a centroid, and adjusting the centroid. Additional clustering techniques may include hierarchical clustering and density based spatial clustering. Hierarchy clustering may be used to identify the groups in the set of candidate drug compounds where there is no set number of clusters to be generated. As a result, a tree-based representation of the objects in the various groups may be generated. Density-based spatial clustering may be used to identify clusters of any shape in a dataset having noise and outliers. This form of clustering also does not require specifying the number of clusters to be generated.

1. A method for identifying agents capable of inducing specific differentiation of pluripotent stem cells into a particular tissue through the utilization of a computing system, said comprising: a) obtaining information regarding cellular and molecular characteristics of an embryonic developing tissue; b) analyzing one or more molecular and/or cellular features of the tissue during stages of differentiation, wherein analyzing the one or more features includes using an artificial intelligence architecture that includes a separable convolutional neural network; and based at least in part on the analysis of the one or more features; and c) obtaining predictive data in the form of associated proteins, extracellular matrix, and nucleic acids that are associated with the process of differentiation.

2. The method of claim 1, which is enabled/designed to create a score for each of the one or more methodologies and/or molecular pathways associated with cellular differentiation, wherein the score reflects the likelihood that an said pathway and/or methodology contains a the necessary information and/or detail to reproducibly generate differentiation of said pluripotent stem cells into a desired tissue and/or organ.

3. The method of claim 1, further comprising indicating whether the one or more molecular pathways and/or methodologies have synergy and/or antagonism.

4. The method of claim 1, wherein the one or more molecular pathways associated with differentiation to a specific tissue comprises a dataset of differentiating factors which are corresponding to a biological sample taken from a single subject.

5. The method of claim 1, wherein said pluripotent stem cell is an inducible pluripotent stem cell.

6. The method of claim 5, wherein said inducible pluripotent stem cell is generated by a mechanical stress associated means.

7. The method of claim 6, wherein said thymus derived cell expresses the marker CD28.

8. The method of claim 6, wherein said thymus derived cell expresses the marker ICOS.

9. The method of claim 6, wherein said thymus derived cell has been treated with a histone deacetylase inhibitor.

10. The method of claim 9, wherein said histone deactylase inhibitor is anexinostat.

11. The method of claim 9, wherein said histone deactylase inhibitor is bg45.

12. The method of claim 5, wherein said cell to be converted to a pluripotent stem cell by said mechanical stress associated means is plated in a droplet that partially or fully surrounds said somatic cell.

13. The method of claim 12, wherein forming the droplet includes pressurized flow through an orifice without heat but with increased atmospheric pressure compared to baseline conditions.

14. The method of claim 13, said increased atmospheric pressure is sufficient to induce activation of janus activated kinase.

15. The method of claim 12, wherein forming the droplet includes using an ultrasonic transducer.

16. The method of claim 12, further comprising printing the pluripotent stem cell on a substrate and incubating the pluripotent stem cell.

17. The method of claim 16, wherein said decellularized matrix is obtained from omental tissue.

18. The method of claim 16, wherein said decellularized matrix is obtained from intestinal mucosal tissue.

19. The method of claim 16, wherein said decellularized matrix is obtained from placental tissue.

20. The method of claim 19, wherein said placental matrix is treated with a Nobel gas prior to utilization.
